(19) United States
(12) Patent Application Publication
(10) Pub. No.: US 2024/0350634 A1
(43) Pub. Date: Oct. 24, 2024

(54) MONOCLONAL ANTIBODY AND VACCINE TARGETING FILAMENTOUS BACTERIOPHAGE

(71) Applicant: INIMMUNE CORPORATION, Missoula, MT (US)

(72) Inventors: Paul L. Bollyky, Stanford, CA (US); William Parks, Seattle, WA (US); Patrick Secor, Seattle, WA (US)

(21) Appl. No.: 18/425,772

(22) Filed: Jan. 29, 2024

Related U.S. Application Data
(63) Continuation of application No. 17/066,005, filed on Oct. 8, 2020, now Pat. No. 11,911,472.
Application No. 17/066,005 is a continuation of application No. 16/262,548, filed on Jan. 30, 2019, now Pat. No. 10,835,607.
Application No. 16/262,548 is a continuation of application No. 15/219,073, filed on Jul. 25, 2016, now abandoned.
(60) Provisional application No. 62/196,147, filed on Jul. 23, 2015.

(51) Int. Cl. A61K 45/06; A61K 39/00; A61K 39/104; A61K 47/64; C07K 16/08

(52) U.S. Cl. CPC A61K 45/06; A61K 39/104; A61K 47/646; C07K 16/08; A61K 39/00; A61K 2039/505; C07K 2317/76

(57) ABSTRACT

Described here is a method for reducing or preventing Pseudomonas aeruginosa biofilm formation in a human subject in need thereof, comprising administering to the human subject a first composition comprising (a) an antigen-binding polypeptide that binds Pf-family bacteriophage, or (b) a vaccine against Pf-family bacteriophage. Also described is an antigen-binding polypeptide that binds specifically to a CoaB protein of Pf-family bacteriophage or fragment thereof.

### CROSS-REFERENCE TO RELATED APPLICATIONS

This application is a continuation of U.S. patent application Ser. No. 17/066,005, filed Oct. 8, 2020, which is a continuation of U.S. patent application Ser. No. 16/262,548, filed on Jan. 30, 2019, now U.S. Pat. No. 10,835,607, which is a continuation of U.S. patent application Ser. No. 15/219,073, filed on Jul. 25, 2016, now abandoned, which claims the benefit of U.S. Provisional Patent Application No. 62/196,147, filed on Jul. 23, 2015, the entire contents of each of which are fully incorporated herein by reference.

### FEDERAL FUNDING STATEMENT

This invention was made with Government support under contract HL007287 awarded by the National Institutes of Health. The Government has certain rights in the invention.

### SEQUENCE LISTING

This application was filed with a Sequence Listing in ST.26 XML format accordance with 37 C.F.R. § 1.831 and PCT Rule 13ter. The Sequence Listing XML file submitted in the USPTO Patent Center, “210468-9004-US03_sequence_listing_XML_9 Jul. 2024.xml,” was created on Jul. 9, 2024, contains 31 sequences, has a file size of 28.0 kilobytes (28,672 bytes), and is incorporated by reference in its entirety into the specification.

### BACKGROUND

Pseudomonas aeruginosa is an opportunistic pathogen in multiple clinical settings, including devastating pulmonary infections in individuals with the genetic disorder cystic fibrosis (CF). The virulence of P. aeruginosa is predicated on its ability to form biofilms, which are organized communities of bacteria encased in a polymer-rich matrix. Bacterial biofilms are ubiquitous in nature, and they endow bacteria with the ability to resist antibiotics and evade host immune defense mechanisms. See Costerton et al., Science 284:1318 (1999); Stewart et al., Int. J. Med. Microbiol. 292:107 (2002).

Thus, a need exists to develop therapeutic agents that can inhibit the formation of P. aeruginosa biofilms thereby making P. aeruginosa infections more susceptible to antibiotic treatment.

### SUMMARY

Many embodiments described herein relate to a method for reducing or preventing P. aeruginosa biofilm formation in a human subject in need thereof, comprising administering to the human subject a first composition comprising (a) an antigen-binding polypeptide that binds Pf-family bacteriophage, or (b) a vaccine against Pf-family bacteriophage.

In one embodiment, the first composition comprises an antigen-binding polypeptide. In one embodiment, the antigen-binding polypeptide binds specifically to a CoaB protein of Pf-family bacteriophage or fragment thereof. In one embodiment, the antigen-binding polypeptide is IgG or IgM.

In one embodiment, the first composition further comprises an antibiotic, or the method further comprises administering a second composition comprising an antibiotic to the human subject.

In one embodiment, the human subject is infected with a P. aeruginosa strain resistant to one or more antibiotics.

In one embodiment, the first composition comprises a vaccine against Pf-family bacteriophage. In one embodiment, the vaccine comprises an immunogenic fragment of Pf-family bacteriophage.

In one embodiment, the CoaB protein or fragment thereof comprises the amino acid sequence of GVIDTSAVESAITDGQGDM (SEQ ID NO: 1).

In one embodiment, the human subject is suffering from cystic fibrosis, burns, chronic would, chronic rhinosinusitis, ventilator-associated pneumonia, catheter-associated urinary tract infections, septic shock, and/or gastrointestinal infections.

Further embodiments described herein relate to an antigen-binding polypeptide that binds specifically to a CoaB protein of Pf-family bacteriophage or fragment thereof.

In one embodiment, the antigen-binding polypeptide specifically binds to an antigenic fragment of the CoaB protein comprising the amino acid sequence of GVIDTSAVESAITDGQGDM (SEQ ID NO: 1).

In one embodiment, the antigen-binding polypeptide is a monoclonal antibody, a chimeric antibody, a humanized antibody, a Fab fragment, a Fab′ fragment, a F(ab′) 2 fragment, or an scFv molecule. In one embodiment, the antigen-binding polypeptide is IgG or IgM.

Another embodiment described herein relates to a pharmaceutical composition comprising (a) the antigen-binding polypeptide described herein or a fusion molecule comprising the antigen-binding polypeptide, and (b) a pharmaceutically acceptable carrier.

Another embodiment described herein relates to a polynucleotide encoding the antigen-binding polypeptide described herein.

Another embodiment described herein relates to an expression cassette comprising a promoter operably linked to the polynucleotide described herein.

Another embodiment described herein relates to a vector comprising the expression cassette described herein.

Another embodiment described herein relates to a transformed cell comprising the expression cassette or the vector described herein.

Another embodiment described herein relates to a method for producing an antigen-binding polypeptide, comprising culturing the transformed cell described herein, and isolating the antigen-binding polypeptide expressed by the transformed cell.

In one embodiment, the methods herein relate to the co-delivery of antibiotics with the antigen-binding polypeptide (e.g., monoclonal antibody) described herein.

Additional embodiments described herein relate to a vaccine against Pf-family bacteriophage, comprising (a) an immunogenic fragment of CoaB protein of Pf-family bacteriophage, and (b) a pharmaceutically acceptable excipient.

In one embodiment, the immunogenic fragment of CoaB protein consists of the amino acid sequence of GVIDTSAVESAITDGQGDM (SEQ ID NO:1)

These and other features, together with the organization and manner of operation thereof, will become apparent from the following detailed description when taken in conjunction with the accompanying drawings.

### BRIEF DESCRIPTION OF THE DRAWINGS

FIG. 1A-1D. Pf 4 bacteriophage organizes P. aeruginosa biofilms into a liquid crystalline structure. FIG. 1A: Pf4 production by colony biofilms formed from the indicated strains enumerated as plaque forming units/milliliter (PFU/ml). Two versions of the standard PA01 P. aeruginosa lab strain were used: a “rough” strain that produces only a modest biofilm and a small colony variant (SCV) strain that produces robust biofilms. In addition, strain PA0728, a version of PA01 that does not produce Pf phage, was also used. Adjusted total Pf phage content are also plotted on the right axis. Results are mean±SD of three experiments. FIG. 1B: Representative images of rough and SCV colony biofilms showing transmitted light (displayed as I/Io) and birefringence (jsin (d) j). FIG. 1C: Birefringence (sin (d)) of the indicated colony biofilms was quantified after normalizing for sample thickness. Birefringence, the capacity of a material to split light into two beams with perpendicular polarization, is a signature characteristic of crystals, Thus, assessments of birefringence can be used to determine whether a biofilm is a crystal. Birefringence was again measured after washing of the bacteria to remove the extracellular matrix. Results are mean±SD of four experiments. FIG. 1D: Representative images for SVC and “rough” colony biofilms (placed between glass plates) visualized through crossed polarizing lenses. Birefringence is visualized as bright areas where light passes through both polarizing lenses. The birefringence patterns change when the sample is rotated with respect to the polarizing lenses, revealing extended areas of birefringence. Together, the data in FIG. 1A-1D show that biofilms made by P. aeruginosa are liquid crystals and that this crystalline organization is dependent on the presence of Pf phage.

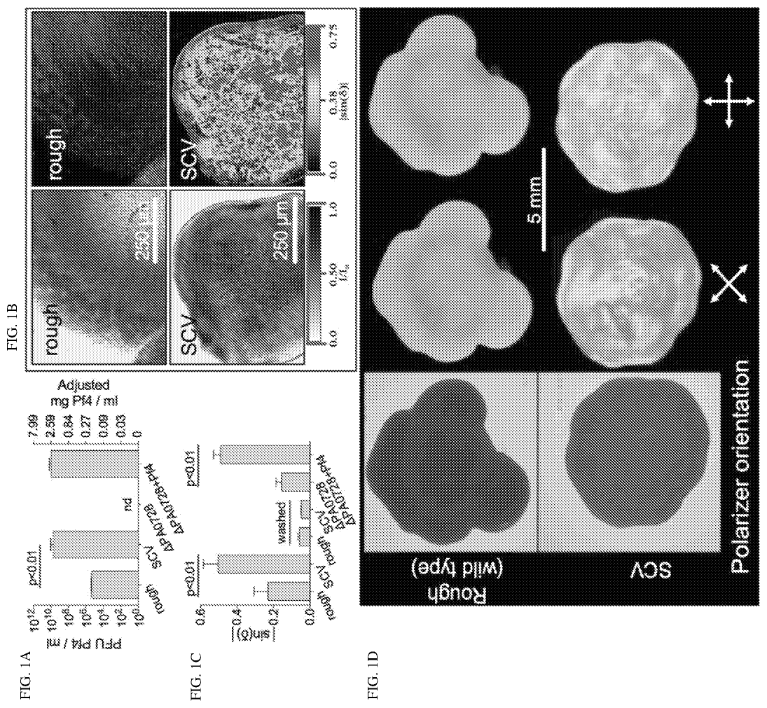

FIG. 2A-2F show that a filamentous bacteriophage, Pf4, contributes to P. aeruginosa biofilm function. FIG. 2A-2F show data describing biofilm characteristics for two strains of P. aeruginosa-PA01 (a standard laboratory strain) and PA0728 (a version of PA01 from which the promoter responsible for Pf4 phage production has been deleted, such that Pf phage production is reduced). In FIG. 2A, the Pf phage copy number is shown for P. aeruginosa strains PA01 and PA0728, as measured by quantitative PCR. In FIG. 2B the amount of CoaB protein, the coat protein that surrounds phage, is shown to be reduced in PA0728 versus PA01 by Mass Spectrometry. In FIG. 2C, the amount of P. aeruginosa colony aggregation is shown to be reduced for PA0728 versus PA01. In FIG. 2D, the adhesion of these bacterial colonies is shown for PA0728 versus PA01, as measured in a flow chamber and crystal violet staining. In FIG. 2E, the susceptibility of PA0728 versus PA01 to 10 μg/ml of the antibiotic gentamycin is shown. In FIG. 2F, the birefringence of PA0728 versus PA01 is shown. It has been recently reported that Pf phage contribute to the organization of P. aeruginosa biofilms into a liquid crystal and that this contributes to biofilm adhesion and antibiotic tolerance. Together, the data in FIG. 2A-2F show that the presence of Pf phage contributes to P. aeruginosa biofilm function. These data are further elaborated upon in Secor et al., Cell Host & Microbe., 18 (5): 549-559 (2015).

FIG. 3 shows that supplementation of P. aeruginosa strain PA01 with Pf4 bacteriophage makes these colonies more tolerant to multiple antibiotics. Killing is represented as the log 10 reduction of viable cells recovered from cultures treated with different antibiotics, all at 10 mg/ml, compared to untreated controls. Results are mean±SD of three experiments.

FIG. 4 shows that serum from rabbits immunized with whole Pf phage prevents biofilm formation. Biofilm formation was assayed by quantification of birefringence (liquid crystal-like organization of the biofilm matrix) of PA01 cultures 18 h after seeding and treatment with serum from rabbits before or after immunization against Pf4 phage.

FIG. 5 shows that the addition of secondary antibodies (IgG) to biofilms pre-treated with antiserum undergo aggregation and clumping. Serum from rabbits before (pre-serum) or after immunization against Pf4 phage (anti-serum) was added to cultures of a strain of PA01. Then, a fluorescently labeled secondary antibody against rabbit IgG was used to visualize the clustering of bacteria. Confocal microscopy images are shown on the Left side of the image while fluorescence microscopy images are shown on the Right.

FIG. 6 shows that the addition of secondary antibodies (IgM) to biofilms pre-treated with antiserum undergo aggregation and clumping. Serum from rabbits before (pre-serum) or after immunization against Pf4 phage (anti-serum) was added to cultures of P. aeruginosa strain PA01. Then, a fluorescently labeled secondary antibody against rabbit IgM was used to visualize the clustering of bacteria. Confocal microscopy images are shown on the Left side of the image while fluorescence microscopy images are shown on the Right.

FIG. 7 shows that monoclonal antibody directed against Pf-family bacteriophage disrupt tactoidal (crystalline) structure formation by Pf4-polymer solutions. For these experiments, a panel of monoclonal antibodies were generated against the coat protein (CoaB) of Pf4 bacteriophage. All of these monoclonal antibodies had similar effects on tactoidal structures.

FIG. 8 shows a semi quantitative dot colony-forming assay that demonstrates that the anti-Pf4 monoclonal antibodies facilitate the penetration of antibiotics into biofilms (exposed to 10 μg/ml of Tobramycin). These experiments were performed by adding one of 4 monoclonal antibodies (AB1, AB2, AB3, AB4) that recognize Pf phage or a control antibody that does not recognize pf phage (AB5) to dilutions of biofilm cultures of the PA01 strain of P. aeruginosa. The size of the bacterial colonies that grow in this setting is inversely related to the penetration of antibiotic through the biofilm.

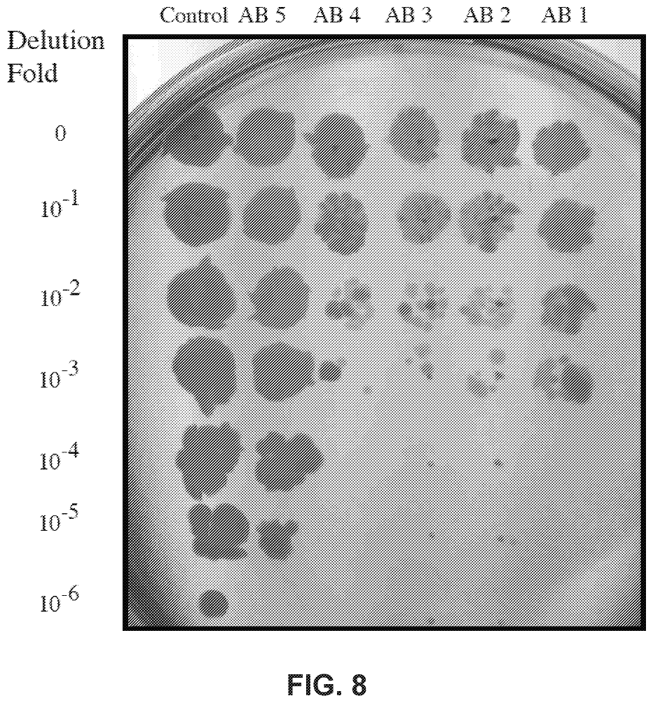

FIG. 9 shows PA concentration survived when exposed to anti-Pf4 antibodies and 10 μg/ml of Tobramycin. This figure provides quantification of the bacterial killing observed in FIG. 8.

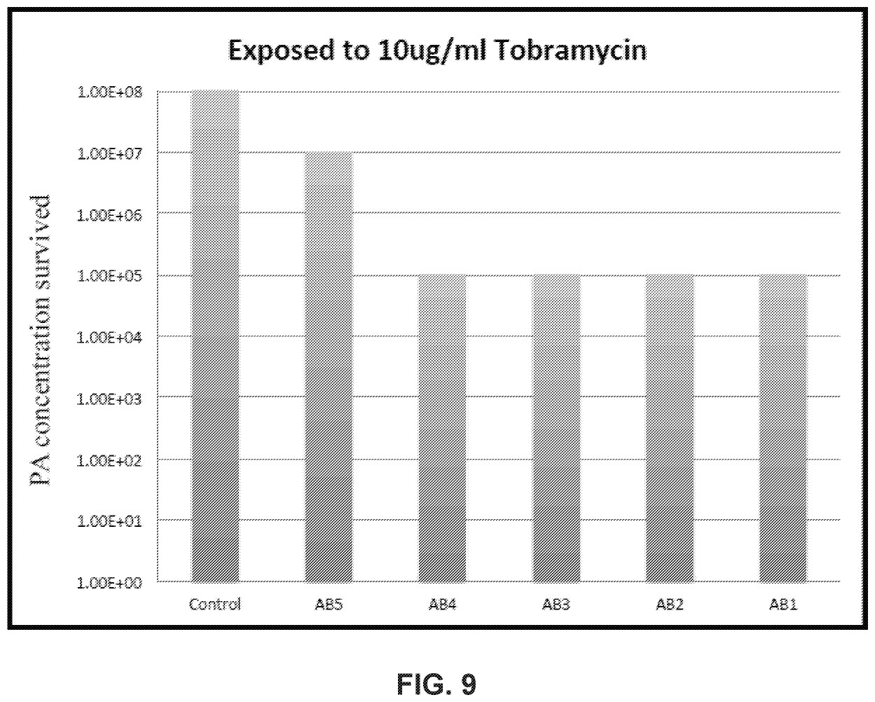

FIG. 10 shows a semi quantitative dot colony-forming assay that demonstrates that the anti-Pf4 antibodies facilitate the penetration of antibiotics into biofilms (exposed to 50 g/ml of Tobramycin). These experiments were performed by adding one of 4 monoclonal antibodies (AB1, AB2, AB3, AB4) that recognize Pf phage or a control antibody that does not recognize pf phage (AB5) to dilutions of biofilm cultures of the PA01 strain of P. aeruginosa. The size of the bacterial colonies that grow in this setting is inversely related to the penetration of antibiotic through the biofilm. Compared to FIG. 8, these data show that the effect of the antibodies was more intense when the biofilms were exposed to higher concentration of Tobramycin (50 ug/ml compared to 10 ug/ml). In that case AB1 and AB2 cause to 7 orders of magnitude reduction in P. aeruginosa concentration that survived the Tobramycin treatment compared to the control.

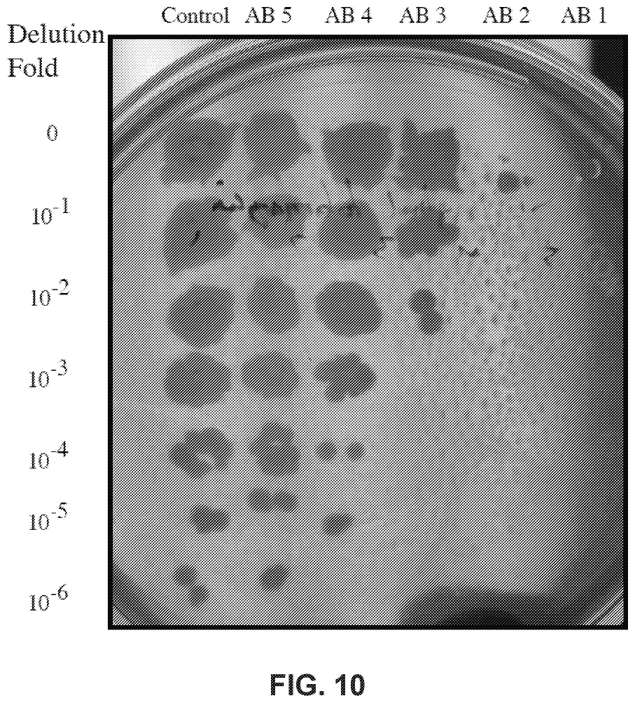

FIG. 11 shows PA concentration survived when exposed to anti-Pf4 antibodies and 50 μg/ml of Tobramycin. This figure provides quantification of the bacterial killing observed in FIG. 10.

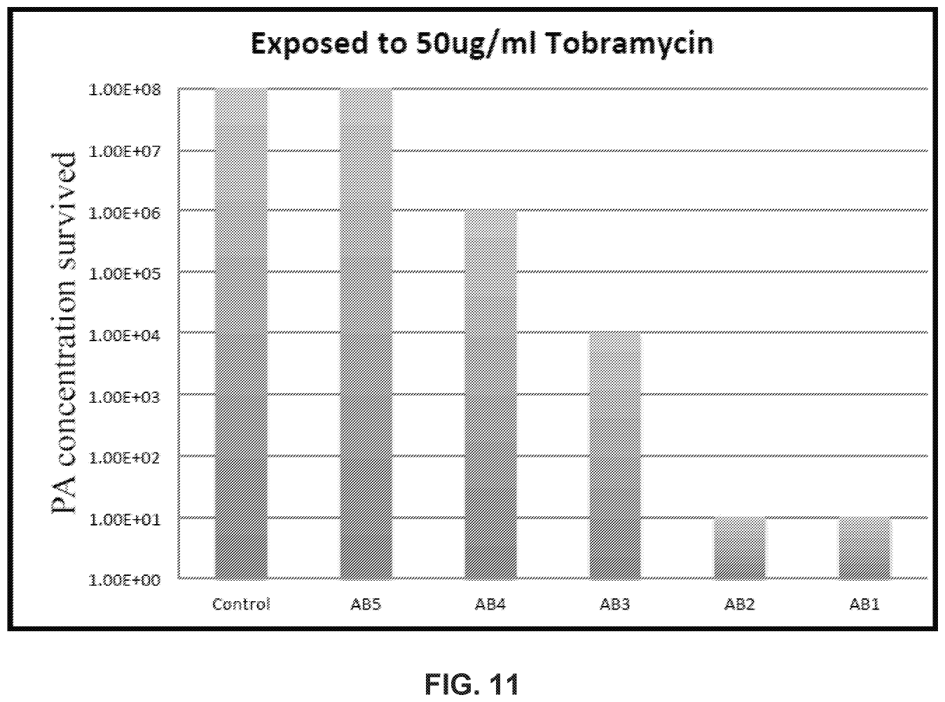

FIG. 12 shows detection of the heavy and light chains of 5 different monoclonal antibodies by COOMASSIE staining and by Western Blot (WB). Three of the antibodies are IgG and two of the antibodies are IgM. All of these antibodies were generated against the Pf phage coat protein sequence GVIDTSAVESAITDGQGDM (SEQ ID NO: 1).

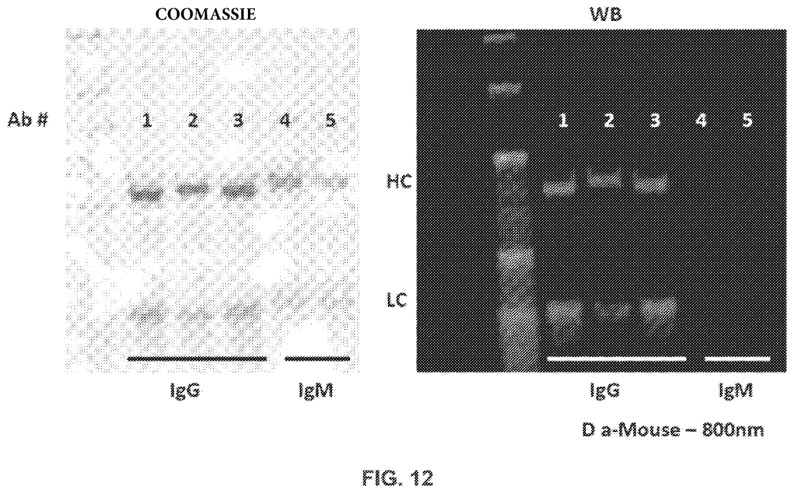

FIG. 13 shows detection of the CoaB coat protein of Pf bacteriophage by 5 different monoclonal antibodies by Western Blot (WB). Three of the antibodies are IgG and two of the antibodies are IgM. One of the IgM (Ab5) does not recognize Pf4 phage and is included here as a control.

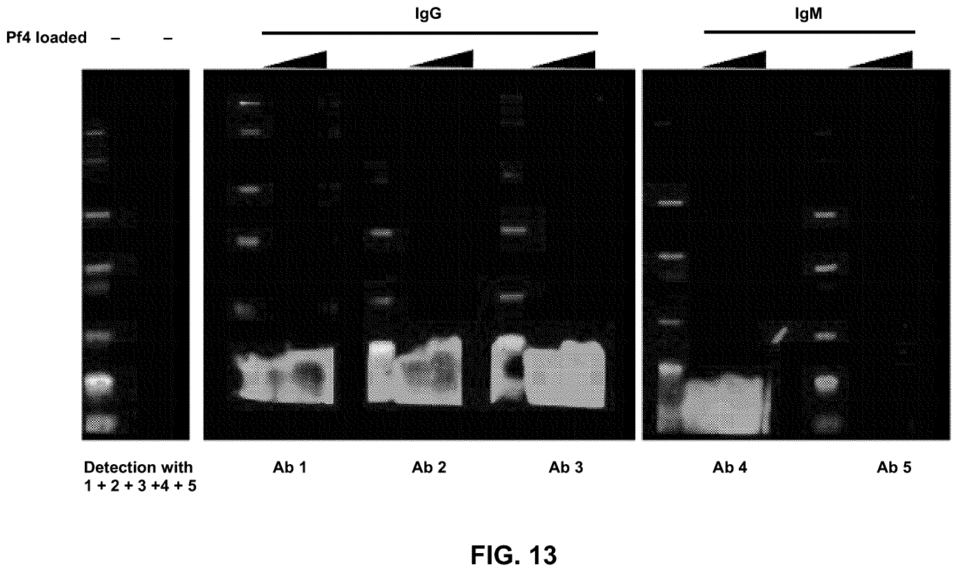

### DETAILED DESCRIPTION

### Introduction

Pf-family bacteriophage play a heretofore unappreciated role in microbial biofilm formation. Monoclonal antibodies or immunizations directed against Pf-family bacteriophage provide protection against biofilm-associated infections with P. aeruginosa and other microbial pathogens. In particular, P. aeruginosa and other Gram-negative microbial pathogens can be resistant to multiple antibiotics, which is an enormous medical problem. Described herein are monoclonal antibodies and immunizations directed against Pf-family bacteriophage which can prevent biofilm formation by these microbes. Therefore, these monoclonal antibodies are an effective class of antibiotic.

In some embodiments, described herein are monoclonal antibodies (both IgG and IgM) directed against conserved regions on the CoaB coat protein of Pf-family bacteriophage. In other embodiments, described herein are synthetic peptides of these same conserved regions on the CoaB coat protein that can be used to immunize animals and human beings against Pf-family bacteriophage. Details of the methods and therapeutic agents of the present invention are provided in the following paragraphs.

Method For Treating/Preventing P. aeruginosa Infection

One aspect of the present invention relates to a method for treating or preventing P. aeruginosa infection in a human subject, which can be achieved by, for example, reducing or preventing P. aeruginosa biofilm formation. The method can comprise, for example, administering to the human subject a composition comprising (a) an antigen-binding polypeptide that binds Pf-family bacteriophage, or (b) a vaccine against Pf-family bacteriophage.

The human subject can be administered with, for example, one or more antigen-binding polypeptides as described herein. The antigen-binding polypeptide can be, for example, an antibody such as IgG or IgM. The antigen-binding polypeptide can bind specifically to, for example, a CoaB protein of Pf-family bacteriophage or fragment thereof. The CoaB protein or fragment thereof can be, for example, the CoaB protein of Pf4 bacteriophage. The CoaB protein or fragment thereof can comprise the amino acid sequence of, for example, GVIDTSAVESAITDGQGDM (SEQ ID NO:1).

The human subject can be administered with, for example, one or more antibiotics. The antibiotics can comprise a known antibiotic against P. aeruginosa, which includes but is not limited to, Aminoglycosides (including for example, tobramycin and gentamicin), Cephalosporins (including ceftazidime), and flouroquinolones (including ciprofloxacin). The antibiotics can be administered to the human subject either sequentially or simultaneously with the antigen-binding polypeptides. Without being bound by any theory, it is believed that the antigen-binding polypeptide could reduce or inhibit P. aeruginosa biofilm formation, thereby rendering the P. aeruginosa more susceptible to antibiotics.

The human subject can be, for example, infected with a P. aeruginosa strain resistant to one or more antibiotics. The human subject can be, for example, infected with one or more additional gram negative pathogens.

The human subject can be, for example, suffering from cystic fibrosis. The human subject can be, for example, suffering from burns. The human subject can be, for example, suffering from chronic wounds. The human subject can be, for example, suffering from chronic rhinosinusitis. The human subject can be, for example, suffering from ventilator-associated pneumonia. The human subject can be, for example, suffering from catheter-associated urinary tract infections. The human subject can be, for example, suffering from septic shock. The human subject can be, for example, suffering from gastrointestinal infections.

The human subject can be administered with, for example, a vaccine against Pf-family bacteriophage, such as Pf4 bacteriophage. The vaccine can comprise, for example, an immunogenic fragment of the CoaB protein of Pf-family bacteriophage as described herein.

The human subject being vaccinated can be, for example, an immune-compromised person, such as a person suffering from AIDS. The human subject being vaccinated can be, for example a person over 60 years old, or a person over 65 years old, or a person over 70 years old.

The vaccination can immunize the human subject against, for example, P. aeruginosa biofilm formation. The vaccination can immunize the human subject against, for example, P. aeruginosa infection.

### Antibody Against Pf-Family Bacteriophage

Another aspect of the present invention relates to an antigen-binding polypeptide that binds specifically to CoaB protein of Pf-family bacteriophage or fragment thereof. The CoaB protein or fragment thereof can be, for example, the CoaB protein of Pf4 bacteriophage. The CoaB protein or fragment thereof can comprise the amino acid sequence of, for example, GVIDTSAVESAITDGQGDM (SEQ ID NO:1).

The antigen-binding polypeptide can be, for example, a monoclonal antibody, a chimeric antibody, a humanized antibody, a Fab fragment, a Fab′ fragment, a F(ab′) 2 fragment, or an scFv molecule. The process for making antigen-binding polypeptides are described in, for example, WO/2008/094942, WO/2009/064854, and WO/2010/111180, which are incorporated by reference in their entireties. In one embodiment, the process comprises (a) immunizing a host with an immunogenic polypeptide of a Pf-family bacteriophage, such as an immunogenic fragment of the CoaB protein of Pf4 bacteriophage; and (b) harvesting the resulting antibody against the immunogenic polypeptide.

The antigen-binding polypeptide can be, for example, part of a fusion molecule. The fusion molecule can comprise, for example, a therapeutic or diagnostic agent conjugated to the antigen-binding polypeptide, as described in WO/2008/094942, WO/2009/064854, and WO/2010/111180.

The monoclonal antibodies can also be functionalized to better disrupt biofilms. The functionalized monoclonal antibodies can comprise, for example, enzymes that degrade constituents of the biofilm matrix, such as DNase I or alginate lyase, or charged molecules such as QDOTs or latex beads intended to alter the tertiary structure of the biofilm matrix. Functionalization can also consist of antibiotics, opsonins, reporter molecules, adjuvants, immunogens, or other proteins, carbohydrates or lipids conjugated to the antibodies.

In some embodiments, the antigen-binding polypeptide is an anti-Pf4 monoclonal antibody or fragment thereof. In some embodiments, the monoclonal antibody or fragment thereof specifically binds to the CoaB protein of Pf4 bacteriophage. In some embodiments, the monoclonal antibody or fragment thereof specifically binds to an antigenic fragment of the CoaB protein comprising, consisting essentially of or consisting of the amino acid sequence of GVIDTSAVESAITDGQGDM (SEQ ID NO:1). In some embodiments, the monoclonal antibody or fragment thereof has a humanized heavy chain variable region and a humanized light chain variable region.

In some embodiments, the anti-Pf4 monoclonal antibody or fragment thereof comprises a heavy chain variable region comprising (1) the CDR-H1 comprising, consisting essentially of or consisting of the amino acid sequence of GFTFSSYV (SEQ ID NO: 6); (2) the CDR-H2 comprising, consisting essentially of or consisting of the amino acid sequence of ISSGGST (SEQ ID NO: 7); and (3) the CDR-H3 comprising, consisting essentially of or consisting of the amino acid sequence of LRGQDYGAAY (SEQ ID NO: 8).

In some embodiments, the anti-Pf4 monoclonal antibody or fragment thereof comprises a heavy chain variable region comprising (1) the CDR-H1 comprising, consisting essentially of or consisting of the amino acid sequence of GYSFTSYW (SEQ ID NO: 16); (2) the CDR-H2 comprising, consisting essentially of or consisting of the amino acid sequence of IYPGNSDT (SEQ ID NO: 17); and (3) the CDR-H3 comprising, consisting essentially of or consisting of the amino acid sequence of TRSQFYSGSSEDAMDY (SEQ ID NO: 18).

In some embodiments, the anti-Pf4 monoclonal antibody or fragment thereof comprises a heavy chain variable region comprising (1) the CDR-H1 comprising, consisting essentially of or consisting of the amino acid sequence of GYTFTNYG (SEQ ID NO: 26); (2) the CDR-H2 comprising, consisting essentially of or consisting of the amino acid sequence of INTNTGEP (SEQ ID NO: 27); and (3) the CDR-H3 comprising, consisting essentially of or consisting of the amino acid sequence of ARKDYRYWFAY (SEQ ID NO: 28).

In some embodiments, the anti-Pf4 monoclonal antibody or fragment thereof comprises a light chain variable region comprising (1) the CDR-L1 comprising, consisting essentially of or consisting of the amino acid sequence of QSLLDSDGKTY (SEQ ID NO: 9); (2) the CDR-L2 comprising, consisting essentially of or consisting of the amino acid sequence of LVS (SEQ ID NO: 10); and (3) the CDR-L3 comprising, consisting essentially of or consisting of the amino acid sequence of WOGTHEPOT (SEQ ID NO: 11).

In some embodiments, the anti-Pf4 monoclonal antibody or fragment thereof comprises a light chain variable region comprising (1) the CDR-L1 comprising, consisting essentially of or consisting of the amino acid sequence of QSIVHSNGNTY (SEQ ID NO: 19); (2) the CDR-L2 comprising, consisting essentially of or consisting of the amino acid sequence of KVS (SEQ ID NO: 20); and (3) the CDR-L3 comprising, consisting essentially of or consisting of the amino acid sequence of FQGSHVPWT (SEQ ID NO: 21).

In some embodiments, the anti-Pf4 monoclonal antibody or fragment thereof comprises a light chain variable region comprising (1) the CDR-L1 comprising, consisting essentially of or consisting of the amino acid sequence of QSIVHSNGNTY (SEQ ID NO: 29); (2) the CDR-L2 comprising, consisting essentially of or consisting of the amino acid sequence of KVS (SEQ ID NO: 30); and (3) the CDR-L3 comprising, consisting essentially of or consisting of the amino acid sequence of FOGSHVPFT (SEQ ID NO: 31).

In some embodiments, the anti-Pf4 monoclonal antibody or fragment thereof comprises (a) a heavy chain variable region comprising (1) the CDR-H1 comprising, consisting essentially of or consisting of the amino acid sequence of GFTFSSYV (SEQ ID NO: 6); (2) the CDR-H2 comprising, consisting essentially of or consisting of the amino acid sequence of ISSGGST (SEQ ID NO: 7); and (3) the CDR-H3 comprising, consisting essentially of or consisting of the amino acid sequence of LRGQDYGAAY (SEQ ID NO: 8), and (b) a light chain variable region comprising (1) the CDR-L1 comprising, consisting essentially of or consisting of the amino acid sequence of QSLLDSDGKTY (SEQ ID NO: 9); (2) the CDR-L2 comprising, consisting essentially of or consisting of the amino acid sequence of LVS (SEQ ID NO: 10); and (3) the CDR-L3 comprising, consisting essentially of or consisting of the amino acid sequence of WOGTHEPOT (SEQ ID NO: 11).

In some embodiments, the anti-Pf4 monoclonal antibody or fragment thereof comprises (a) a heavy chain variable region comprising (1) the CDR-H1 comprising, consisting essentially of or consisting of the amino acid sequence of GYSFTSYW (SEQ ID NO: 16); (2) the CDR-H2 comprising, consisting essentially of or consisting of the amino acid sequence of IYPGNSDT (SEQ ID NO: 17); and (3) the CDR-H3 comprising, consisting essentially of or consisting of the amino acid sequence of TRSQFYSGSSEDAMDY (SEQ ID NO: 18), and (b) a light chain variable region comprising (1) the CDR-L1 comprising, consisting essentially of or consisting of the amino acid sequence of QSIVHSNGNTY (SEQ ID NO: 19); (2) the CDR-L2 comprising, consisting essentially of or consisting of the amino acid sequence of KVS (SEQ ID NO: 20); and (3) the CDR-L3 comprising, consisting essentially of or consisting of the amino acid sequence of FQGSHVPWT (SEQ ID NO: 21).

In some embodiments, the anti-Pf4 monoclonal antibody or fragment thereof comprises (a) a heavy chain variable region comprising (1) the CDR-H1 comprising, consisting essentially of or consisting of the amino acid sequence of GYTFTNYG (SEQ ID NO: 26); (2) the CDR-H2 comprising, consisting essentially of or consisting of the amino acid sequence of INTNTGEP (SEQ ID NO: 27); and (3) the CDR-H3 comprising, consisting essentially of or consisting of the amino acid sequence of ARKDYRYWFAY (SEQ ID NO: 28), and (b) a light chain variable region comprising (1) the CDR-L1 comprising, consisting essentially of or consisting of the amino acid sequence of QSIVHSNGNTY (SEQ ID NO: 29); (2) the CDR-L2 comprising, consisting essentially of or consisting of the amino acid sequence of KVS (SEQ ID NO: 30); and (3) the CDR-L3 comprising, consisting essentially of or consisting of the amino acid sequence of FQGSHVPFT (SEQ ID NO: 31).

In some embodiments, the anti-Pf4 monoclonal antibody or fragment thereof comprises a heavy chain variable region comprising, consisting essentially of or consisting of the amino acid sequence of EVKLVESGGDLVKPGGSLKLSCAASGFTFSSYVMSWVRQTPEKRLEWVASISSGGSTYYPDSVKGRFTIS RDNARNILYLQMSSLRSEDTAMYYCLRGQDYGAAYWGQGTLVTVSA (SEQ ID NO: 2) or a humanized version thereof.

In some embodiments, the anti-Pf4 monoclonal antibody or fragment thereof comprises a heavy chain variable region comprising, consisting essentially of or consisting of the amino acid sequence of EVQLQQSGTVLARPGASVKMSCKASGYSFTSYWMHWVKQRPGQGLEWIGAIYPGNSDTSYNQKFKGKAKL TAVTSASTAYMELSCLTNEDSAVFYCTRSQFYSGSSEDAMDYWGQGTSVTVSS (SEQ ID NO: 12) or a humanized version thereof.

In some embodiments, the anti-Pf4 monoclonal antibody or fragment thereof comprises a heavy chain variable region comprising, consisting essentially of or consisting of the amino acid sequence of QIQLVQSGPELKKPGETVKISCKASGYTFTNYGMNWLKQAPGKGLKWMGWINTNTGEPTYAEEFKGRFAF SLETSASTAYLQINNLKNEDTATYFCARKDYRYWFAYWGQGTLVTVSA (SEQ ID NO: 22) or a humanized version thereof.

In some embodiments, the anti-Pf4 monoclonal antibody or fragment thereof comprises a light chain variable region comprising, consisting essentially of or consisting of the amino acid sequence of DVVMTQTPLTLSVTIGQPASISCKSSQSLLDSDGKTYLNWLLQRPGQSPKRLIYLVSKLDSGVPDRFTGS GSGTDFTLKISRVEAEDLGVYYCWQGTHEPQTFGGGTKLEIK (SEQ ID NO: 4) or a humanized version thereof.

In some embodiments, the anti-Pf4 monoclonal antibody or fragment thereof comprises a light chain variable region comprising, consisting essentially of or consisting of the amino acid sequence of DVLMTQTPLSLPVSLGDQASISCRSSQSIVHSNGNTYLEWYLQKPGQSPKLLIYKVSNRFSGVPDRESGS GSGTDFTLKISRVEAEDLGVY FCFQGSHVPWTFGGGTKLEIK (SEQ ID NO: 14) or a humanized version thereof.

In some embodiments, the anti-Pf4 monoclonal antibody or fragment thereof comprises a light chain variable region comprising, consisting essentially of or consisting of the amino acid sequence of DVLMTQTPLSLPVSLGDQASI SCRSSQSIVHSNGNTYLEWYLQKPGQSPKLLIYKVSNRFSGVPDRESGS GSGTDFTLKISRVEAEDLGVYYCFQGSHVPFTFGSGTKLEIK (SEQ ID NO: 24) or a humanized version thereof.

Another aspect of the present invention relates to a pharmaceutical composition comprising (a) the antigen-binding polypeptide described herein or a fusion molecule comprising the antigen-binding polypeptide, and (b) a pharmaceutically acceptable carrier. In one embodiment, the active ingredient of the pharmaceutical composition consists essentially of the antigen-binding polypeptide or fusion molecule. In another embodiment, the active ingredient of the pharmaceutical composition consists of the antigen-binding polypeptide or fusion molecule.

Another aspect of the present invention relates to a polynucleotide encoding the antigen-binding polypeptide described herein. The polynucleotide encoding the antigen-binding polypeptide can be comprised in, for example, an expression cassette, with optionally a promoter operably linked to the polynucleotide. The expression cassette can be comprised in, for example, a plasmid or transformation vector. The plasmid or transformation vector can be used to obtain a transformed cell capable of producing the antigen-binding polypeptide encoded therein.

In some embodiments, the polynucleotide encodes a heavy chain variable region comprising (1) the CDR-H1 comprising, consisting essentially of or consisting of the amino acid sequence of GFTFSSYV (SEQ ID NO: 6); (2) the CDR-H2 comprising, consisting essentially of or consisting of the amino acid sequence of ISSGGST (SEQ ID NO: 7); and (3) the CDR-H3 comprising, consisting essentially of or consisting of the amino acid sequence of LRGQDYGAAY (SEQ ID NO: 8).

In some embodiments, the polynucleotide encodes a heavy chain variable region comprising (1) the CDR-H1 comprising, consisting essentially of or consisting of the amino acid sequence of GYSFTSYW (SEQ ID NO: 16); (2) the CDR-H2 comprising, consisting essentially of or consisting of the amino acid sequence of IYPGNSDT (SEQ ID NO: 17); and (3) the CDR-H3 comprising, consisting essentially of or consisting of the amino acid sequence of TRSQFYSGSSEDAMDY (SEQ ID NO: 18).

In some embodiments, the polynucleotide encodes a heavy chain variable region comprising (1) the CDR-H1 comprising, consisting essentially of or consisting of the amino acid sequence of GYTFTNYG (SEQ ID NO: 26); (2) the CDR-H2 comprising, consisting essentially of or consisting of the amino acid sequence of INTNTGEP (SEQ ID NO: 27); and (3) the CDR-H3 comprising, consisting essentially of or consisting of the amino acid sequence of ARKDYRYWFAY (SEQ ID NO: 28).

In some embodiments, the polynucleotide encodes a light chain variable region comprising (1) the CDR-L1 comprising, consisting essentially of or consisting of the amino acid sequence of QSLLDSDGKTY (SEQ ID NO: 9); (2) the CDR-L2 comprising, consisting essentially of or consisting of the amino acid sequence of LVS (SEQ ID NO: 10); and (3) the CDR-L3 comprising, consisting essentially of or consisting of the amino acid sequence of WQGTHFPQT (SEQ ID NO: 11).

In some embodiments, the polynucleotide encodes a light chain variable region comprising (1) the CDR-L1 comprising, consisting essentially of or consisting of the amino acid sequence of QSIVHSNGNTY (SEQ ID NO: 19); (2) the CDR-L2 comprising, consisting essentially of or consisting of the amino acid sequence of KVS (SEQ ID NO: 20); and (3) the CDR-L3 comprising, consisting essentially of or consisting of the amino acid sequence of FQGSHVPWT (SEQ ID NO: 21).

In some embodiments, the polynucleotide encodes a light chain variable region comprising (1) the CDR-L1 comprising, consisting essentially of or consisting of the amino acid sequence of QSIVHSNGNTY (SEQ ID NO: 29); (2) the CDR-L2 comprising, consisting essentially of or consisting of the amino acid sequence of KVS (SEQ ID NO: 30); and (3) the CDR-L3 comprising, consisting essentially of or consisting of the amino acid sequence of FOGSHVPFT (SEQ ID NO: 31).

In some embodiments, the polynucleotide encodes (a) a heavy chain variable region comprising (1) the CDR-H1 comprising, consisting essentially of or consisting of the amino acid sequence of GFTFSSYV (SEQ ID NO: 6); (2) the CDR-H2 comprising, consisting essentially of or consisting of the amino acid sequence of ISSGGST (SEQ ID NO: 7); and (3) the CDR-H3 comprising, consisting essentially of or consisting of the amino acid sequence of LRGQDYGAAY (SEQ ID NO: 8), and (b) a light chain variable region comprising (1) the CDR-L1 comprising, consisting essentially of or consisting of the amino acid sequence of QSLLDSDGKTY (SEQ ID NO: 9); (2) the CDR-L2 comprising, consisting essentially of or consisting of the amino acid sequence of LVS (SEQ ID NO: 10); and (3) the CDR-L3 comprising, consisting essentially of or consisting of the amino acid sequence of WQGTHEPOT (SEQ ID NO: 11).

In some embodiments, the polynucleotide encodes (a) a heavy chain variable region comprising (1) the CDR-H1 comprising, consisting essentially of or consisting of the amino acid sequence of GYSFTSYW (SEQ ID NO: 16); (2) the CDR-H2 comprising, consisting essentially of or consisting of the amino acid sequence of IYPGNSDT (SEQ ID NO: 17); and (3) the CDR-H3 comprising, consisting essentially of or consisting of the amino acid sequence of TRSQFYSGSSEDAMDY (SEQ ID NO: 18), and (b) a light chain variable region comprising (1) the CDR-L1 comprising, consisting essentially of or consisting of the amino acid sequence of QSIVHSNGNTY (SEQ ID NO: 19); (2) the CDR-L2 comprising, consisting essentially of or consisting of the amino acid sequence of KVS (SEQ ID NO: 20); and (3) the CDR-L3 comprising, consisting essentially of or consisting of the amino acid sequence of FQGSHVPWT (SEQ ID NO: 21).

In some embodiments, the polynucleotide encodes (a) a heavy chain variable region comprising (1) the CDR-H1 comprising, consisting essentially of or consisting of the amino acid sequence of GYTFTNYG (SEQ ID NO: 26); (2) the CDR-H2 comprising, consisting essentially of or consisting of the amino acid sequence of INTNTGEP (SEQ ID NO: 27); and (3) the CDR-H3 comprising, consisting essentially of or consisting of the amino acid sequence of ARKDYRYWFAY (SEQ ID NO: 28), and (b) a light chain variable region comprising (1) the CDR-L1 comprising, consisting essentially of or consisting of the amino acid sequence of QSIVHSNGNTY (SEQ ID NO: 29); (2) the CDR-L2 comprising, consisting essentially of or consisting of the amino acid sequence of KVS (SEQ ID NO: 30); and (3) the CDR-L3 comprising, consisting essentially of or consisting of the amino acid sequence of FOGSHVPFT (SEQ ID NO: 31).

In some embodiments, the polynucleotide encodes a heavy chain variable region comprising, consisting essentially of or consisting of the amino acid sequence of EVKLVESGGDLVKPGGSLKLSCAASGFTFSSYVMSWVRQTPEKRLEWVASISSGGSTYYPDSVKGRFTIS RDNARNILYLQMSSLRSEDTAMYYCLRGQDYGAAYWGQGTLVTVSA (SEQ ID NO: 2) or a humanized version thereof.

In some embodiments, the polynucleotide encodes a heavy chain variable region comprising, consisting essentially of or consisting of the amino acid sequence of EVQLQQSGTVLARPGASVKMSCKASGYSFTSYWMHWVKQRPGQGLEWIGAIYPGNSDTSYNQKFKGKAKL TAVTSASTAYMELSCLTNEDSAVFYCTRSQFYSGSSEDAMDYWGQGTSVTVSS (SEQ ID NO: 12) or a humanized version thereof.

In some embodiments, the polynucleotide encodes a heavy chain variable region comprising, consisting essentially of or consisting of the amino acid sequence of QIQLVQSGPELKKPGETVKISCKASGYTFTNYGMNWLKQAPGKGLKWMGWINTNTGEPTYAEEFKGRFAF SLETSASTAYLQINNLKNEDTATYFCARKDYRYWFAYWGQGTLVTVSA (SEQ ID NO: 22) or a humanized version thereof.

In some embodiments, the polynucleotide encodes a light chain variable region comprising, consisting essentially of or consisting of the amino acid sequence of DVVMTQTPLTLSVTIGQPASISCKSSQSLLDSDGKTYLNWLLQRPGQSPKRLIYLVSKLDSGVPDRFTGS GSGTDFTLKISRVEAEDLGVYYCWQGTHFPQTFGGGTKLEIK (SEQ ID NO: 4) or a humanized version thereof.

In some embodiments, the polynucleotide encodes a light chain variable region comprising, consisting essentially of or consisting of the amino acid sequence of DVLMTQTPLSLPVSLGDQASISCRSSQSIVHSNGNTYLEWYLQKPGQSPKLLIYKVSNRFSGVPDRESGS GSGTDFTLKISRVEAEDLGVYFCFQGSHVPWTFGGGTKLEIK (SEQ ID NO: 14) or a humanized version thereof.

In some embodiments, the polynucleotide encodes a light chain variable region comprising, consisting essentially of or consisting of the amino acid sequence of DVLMTQTPLSLPVSLGDQASISCRSSQSIVHSNGNTYLEWYLQKPGQSPKLLIYKVSNRFSGVPDRESGS GSGTDFTLKISRVEAEDLGVYYCFQGSHVPFTFGSGTKLEIK (SEQ ID NO: 24) or a humanized version thereof.

### Vaccine Against Pf-Family Bacteriophage

A further aspect of the present invention relates to a vaccine against Pf-family bacteriophage, comprising an immunogenic fragment of the CoaB protein of Pf-family bacteriophage. The CoaB protein or fragment thereof can be, for example, the CoaB protein of Pf4 bacteriophage. The CoaB protein or fragment thereof can comprise the amino acid sequence of, for example, GVIDTSAVESAITDGQGDM (SEQ ID NO:1).

In some embodiments, the vaccine is used to vaccinate patients newly diagnosed with cystic fibrosis before they become colonized with P. aeruginosa or to elderly people before they become prone to catheter infections and hospital-acquired infections. In some embodiments, the vaccine is used to vaccinate nursing home populations, or patients undergoing dialysis, mechanical ventilation or recurrent UTIs, or burn victims.

These and other features, together with the organization and manner of operation thereof, will become apparent from the following detailed description when taken in conjunction with the accompanying drawings.

### Working Examples

It has been recently reported that a bacteriophage/virus produced by the bacteria Pseudomonas aeruginosa contributes to the formation and function of Pseudomonas aeruginosa biofilms. In particular, it has been reported that Pf bacteriophage assemble biofilms into a liquid crystal and that this crystalline organization contributes to biofilm function, including adhesiveness and antibiotic tolerance. See Secor et al., Cell Host & Microbe., 18 (5): 549-559 (2015). In light of these data implicating Pf bacteriophages in biofilm structure and function, vaccines and monoclonal antibodies that target Pf bacteriophages have been generated to disrupt Pseudomonas aeruginosa biofilms.

### Example 1—Role of Pf-Family Phage in Biofilm Formation

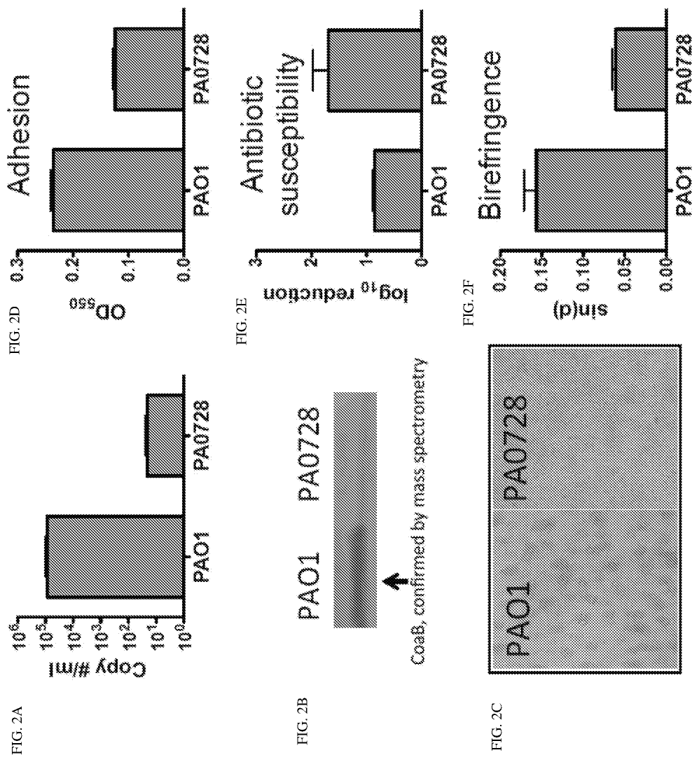

Filamentous bacteriophage are produced by P. aeruginosa during the biofilm mode of growth (see Rice et al., The ISME Journal (2009) 3, 271-282). A mutant not capable of producing Pf4, PA0728 (FIGS. 2A and 2B) (see Castang and Dove, J Bacteriol. September 2012; 194 (18): 5101-5109), exhibited differences in morphology (FIG. 2C), adhesion (FIG. 2D), antibiotic susceptibility (FIG. 2E), and matrix organization (birefringence, FIG. 2F).

### Example 2—Inhibition of Biofilms by Antiserum

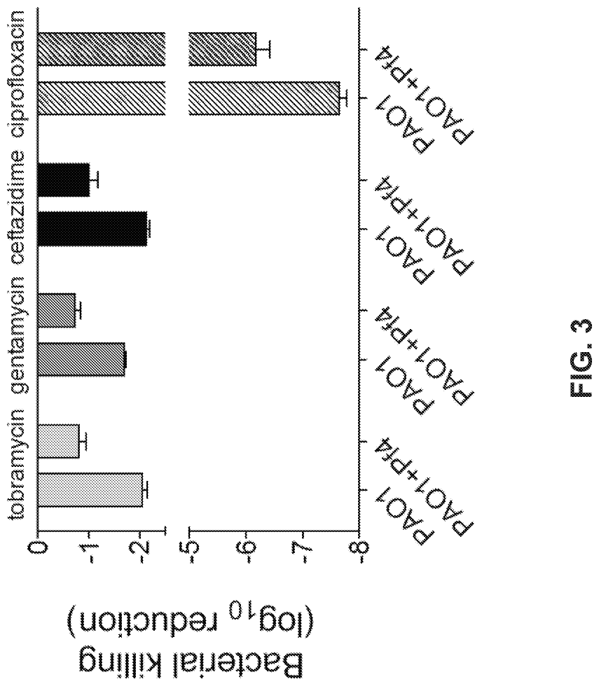

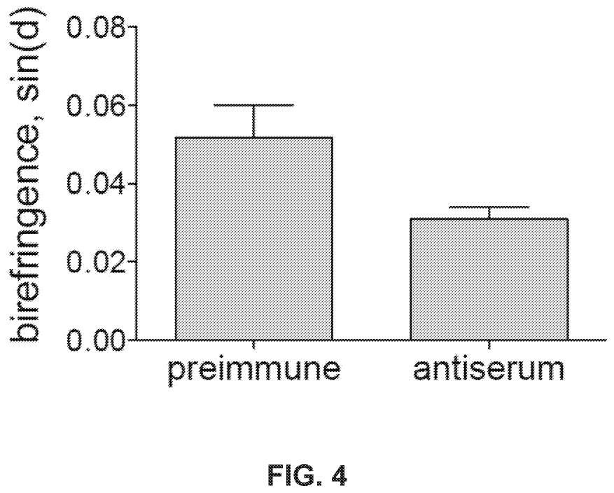

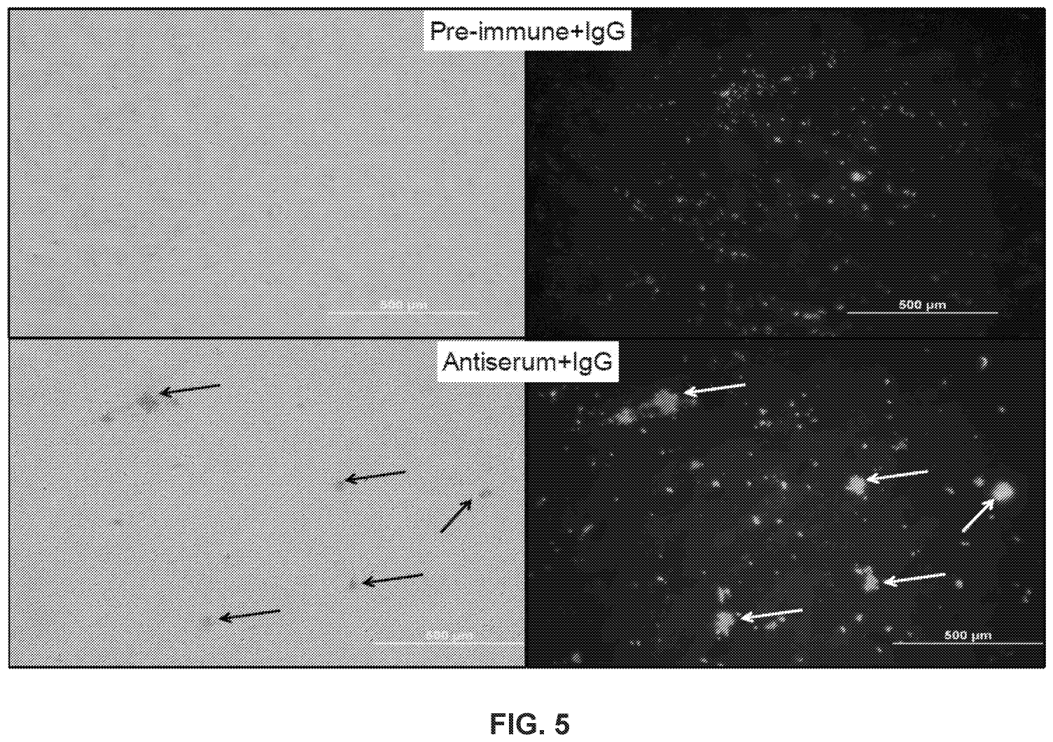

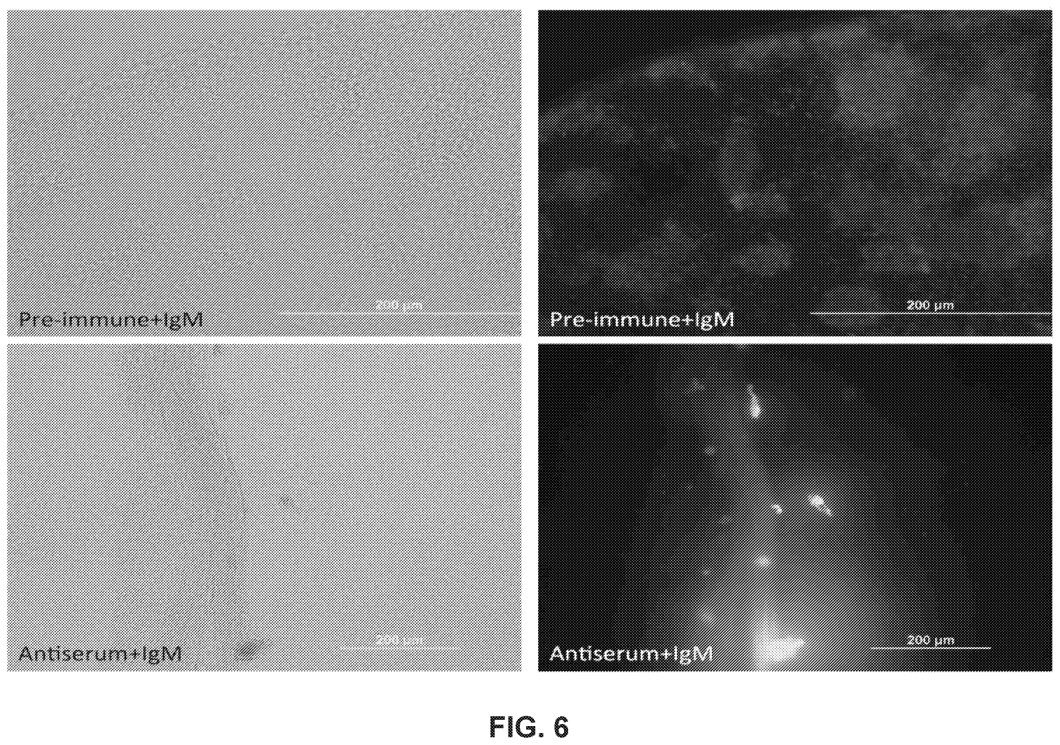

The method of preventing biofilm formation by creating vaccines and monoclonal antibodies that target Pf bacteriophage was evaluated. Specifically, rabbits were immunized with CoaB peptide, and the ability of their sera to neutralize biofilm formation was examined. This antiserum was found to inhibit biofilm formation, alter antibiotic tolerance (FIG. 3), and alter the overall organization of the biofilm matrix (reduced birefringence, FIG. 4), suggesting that anti-CoaB antibodies present in the serum are capable of disrupting P. aeruginosa biofilms producing the filamentous bacteriophage Pf4. The addition of secondary antibodies (e.g., fluorescently labeled anti-rabbit IgG and IgM) to P. aeruginosa biofilms pre-treated with anti-Pf4 antibodies altered the gross morphology of P. aeruginosa biofilms (FIGS. 5 and 6).

### Example 3—Production of Monoclonal Antibody

Monoclonal antibodies that target Pf4, including three IgG and two IgM antibodies, were developed as a new class of anti-microbial for use against multi-drug resistant P. aeruginosa. These antibodies were generated using standard, well-established techniques, generally as described in hypertext transfer protocol www.currentprotocols.com/WileyCDA/CPUnit/refld-im0205.html. In brief, the CoaB peptide in question was conjugated to immunogenic proteins in order to elicit an immune response in rabbits and/or mice. Clones were then isolated from individual animals with strong serum responses to the peptide in question and these were fused to competent cells to generate hybridomas. These were then grown in culture and monoclonal antibodies were harvested from the cell culture supernatants. The specificity of these against CoaB peptide was then confirmed and five clones with the greatest specificity were selected for further development.

The variable regions of the three mouse IgG anti-Pf4 monoclonal antibodies were sequenced. The VH, VL, and CDR sequences of IgG Ab #1 (1A8), IgG Ab #2 (2D4) and IgG Ab #3 (3D6) are provided below.

IgG Ab #1 VH:
(SEQ ID NO: 2)
EVKLVESGGDLVKPGGSLKLSCAASGFTFSSYVMSWVRQTPEKRLEWVAS
ISSGGSTYYPDSVKGRFTISRDNARNILYLQMSSLRSEDTAMYYCLRGQD
YGAAYWGQGTLVTVSA.
(SEQ ID NO: 3)
GAAGTGAAGCTGGTGGAGTCTGGGGGAGACTTAGTGAAGCCTGGAGGGTC
CCTGAAACTCTCCTGTGCAGCCTCTGGATTCACTTTCAGTAGCTATGTCA
TGTCTTGGGTTCGCCAGACTCCAGAAAAGAGGCTGGAGTGGGTCGCATCC
ATTAGTAGTGGTGGTAGCACCTACTATCCAGACAGTGTGAAGGGCCGATT
CACCATCTCCAGAGATAATGCCAGGAACATCCTGTACCTGCAAATGAGTA
GTCTGAGGTCTGAGGACACGGCCATGTATTACTGTTTAAGAGGCCAGGAC
TACGGCGCCGCTTACTGGGGCCAAGGGACTCTGGTCACTGTCTCTGCA.
IgG Ab #1 VL:
(SEQ ID NO: 4)
DVVMTQTPLTLSVTIGQPASISCKSSQSLLDSDGKTYLNWLLQRPGQSPK
RLIYLVSKLDSGVPDRFTGSGSGTDFTLKISRVEAEDLGVYYCWQGTHFP
QTFGGGTKLEIK.
(SEQ ID NO: 5)
GATGTTGTGATGACCCAGACTCCACTCACTTTGTCGGTTACCATTGGACA
ACCAGCCTCCATCTCTTGCAAGTCAAGTCAGAGCCTCTTAGATAGTGATG
GAAAGACATATTTGAATTGGTTGTTACAGAGGCCAGGCCAGTCTCCAAAG
CGCCTAATCTATCTGGTGTCTAAACTGGACTCTGGAGTCCCTGACAGGTT
CACTGGCAGTGGATCAGGGACAGATTTCACACTGAAAATCAGCAGAGTGG
AGGCTGAGGATTTGGGAGTTTATTATTGCTGGCAAGGTACACATTTTCCT
CAGACGTTCGGTGGAGGCACCAAGCTGGAAATCAAA.
IgG Ab #1 CDR:
CDR-H1 -
(SEQ ID NO: 6)
GFTFSSYV.
CDR-H2 -
(SEQ ID NO: 7)
ISSGGST.
CDR-H3 -
(SEQ ID NO: 8)
LRGQDYGAAY.
CDR-L1 -
(SEQ ID NO: 9)
QSLLDSDGKTY.
CDR-L2 -
(SEQ ID NO: 10)
LVS.
CDR-L3 -
(SEQ ID NO: 11)
WQGTHFPQT.
IgG Ab #2 VH:
(SEQ ID NO: 12)
EVQLQQSGTVLARPGASVKMSCKASGYSFTSYWMHWVKQRPGQGLEWIGA
IYPGNSDTSYNQKFKGKAKLTAVTSASTAYMELSCLTNEDSAVFYCTRSQ
FYSGSSEDAMDYWGQGTSVTVSS.
(SEQ ID NO: 13)
GAGGTTCAGCTCCAGCAGTCTGGGACTGTGCTGGCAAGGCCTGGGGCTTC
CGTGAAGATGTCCTGCAAGGCTTCTGGCTACAGCTTTACCAGCTACTGGA
TGCACTGGGTAAAACAGAGGCCTGGACAGGGTCTAGAATGGATTGGTGCT
ATTTATCCTGGAAATAGTGATACTAGTTACAACCAGAAGTTCAAGGGCAA
GGCCAAACTGACTGCAGTCACATCCGCCAGCACTGCCTACATGGAGCTCA
GCTGCCTGACAAATGAGGACTCTGCGGTCTTTTACTGTACAAGATCCCAA
TTTTACTCCGGTAGTAGCGAGGATGCTATGGACTACTGGGGTCAAGGAAC
CTCAGTCACCGTCTCCTCA.
IgG Ab #2 VL:
(SEQ ID NO: 14)
DVLMTQTPLSLPVSLGDQASISCRSSQSIVHSNGNTYLEWYLQKPGQSPK
LLIYKVSNRFSGVPDRFSGSGSGTDFTLKISRVEAEDLGVYFCFQGSHVP
WTFGGGTKLEIK.
(SEQ ID NO: 15)
GATGTTTTGATGACCCAAACTCCACTCTCCCTGCCTGTCAGTCTTGGAGA
TCAAGCCTCCATCTCTTGCAGATCTAGTCAGAGCATTGTACATAGTAATG
GAAACACCTATTTAGAATGGTACCTGCAGAAACCAGGCCAGTCTCCAAAG
CTCCTGATCTACAAAGTTTCCAACCGATTTTCTGGGGTCCCAGACAGGTT
CAGTGGCAGTGGATCAGGGACAGATTTCACACTCAAGATCAGCAGAGTGG
AGGCTGAGGATCTGGGAGTTTATTTCTGCTTTCAAGGTTCACATGTTCCG
TGGACGTTCGGTGGAGGCACCAAGCTGGAAATCAAA.
IgG Ab #2 CDR:
CDR-H1 -
(SEQ ID NO: 16)
GYSFTSYW.
CDR-H2 -
(SEQ ID NO: 17)
IYPGNSDT.
CDR-H3 -
(SEQ ID NO: 18)
TRSQFYSGSSEDAMDY.
CDR-L1 -
(SEQ ID NO: 19)
QSIVHSNGNTY.
CDR-L2 -
(SEQ ID NO: 20)
KVS.
CDR-L3 -
(SEQ ID NO: 21)
FQGSHVPWT.
IgG Ab #3 VH:
(SEQ ID NO: 22)
QIQLVQSGPELKKPGETVKISCKASGYTFTNYGMNWLKQAPGKGLKWMGW
INTNTGEPTYAEEFKGRFAFSLETSASTAYLQINNLKNEDTATYFCARKD
YRYWFAYWGQGTLVTVSA.
(SEQ ID NO: 23)
CAGATCCAGTTGGTGCAGTCTGGACCTGAGCTGAAGAAGCCTGGAGAGAC
AGTCAAGATCTCCTGCAAGGCTTCTGGGTATACCTTCACAAACTATGGAA
TGAACTGGCTGAAGCAGGCTCCAGGAAAGGGTTTAAAGTGGATGGGCTGG
ATAAACACCAACACTGGAGAGCCAACATATGCTGAAGAGTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGAAAGGAC
TATAGGTACTGGTTTGCTTACTGGGGCCAAGGGACTCTGGTCACTGTCTC
TGCA.
IgG Ab #3 VL:
(SEQ ID NO: 24)
DVLMTQTPLSLPVSLGDQASISCRSSQSIVHSNGNTYLEWYLQKPGQSPK
LLIYKVSNRFSGVPDRFSGSGSGTDFTLKISRVEAEDLGVYYCFQGSHVP
FTFGSGTKLEIK.
(SEQ ID NO: 25)
GATGTTTTGATGACCCAAACTCCACTCTCCCTGCCTGTCAGTCTTGGAGA
TCAAGCCTCCATCTCTTGCAGATCTAGTCAGAGCATTGTACATAGTAATG
GAAACACCTATTTAGAATGGTACCTGCAGAAACCAGGCCAGTCTCCAAAG
CTCCTGATCTACAAAGTTTCCAACCGATTTTCTGGGGTCCCAGACAGGTT
CAGTGGCAGTGGATCAGGGACAGATTTCACACTCAAGATCAGCAGAGTGG
AGGCTGAGGATCTGGGAGTTTATTACTGCTTTCAAGGTTCACATGTTCCA
TTCACGTTCGGCTCGGGGACAAAGTTGGAAATAAAA.
IgG Ab #3 CDR:
CDR-H1 -
(SEQ ID NO: 26)
GYTFTNYG.
CDR-H2 -
(SEQ ID NO: 27)
INTNTGEP.
CDR-H3 -
(SEQ ID NO: 28)
ARKDYRYWFAY.
CDR-L1 -
(SEQ ID NO: 29)
QSIVHSNGNTY.
CDR-L2 -
(SEQ ID NO: 30)
KVS.
CDR-L3 -
(SEQ ID NO: 31)
FQGSHVPFT.

### Example 4—Inhibition of Biofilms by Monoclonal Antibody

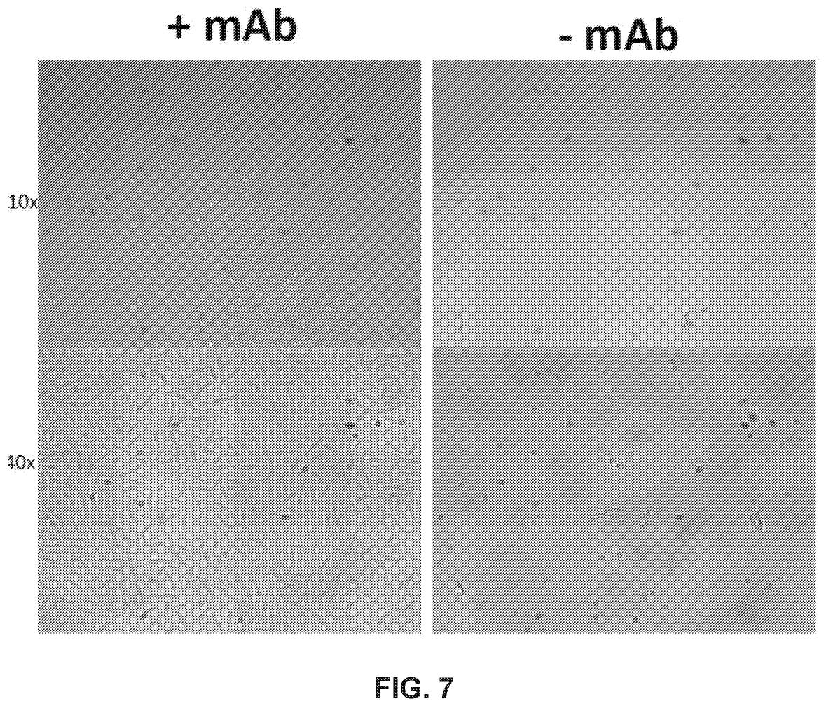

Also tested is whether the monoclonal antibodies produced according to Example 2 can serve as effective antibiotic drugs that could be used in conjunction with conventional antibiotics, as current therapeutic options for treatment of multidrug-resistant P. aeruginosa are very limited. As shown in FIG. 7, the mAbs prevented liquid crystal formation of Pf4-polymer solutions suggesting that the mAbs have the same activities as the anti-Pf4 antibodies present in the antiserum of Example 2.

As used herein, the singular terms “a,” “an,” and “the” include plural referents unless the context clearly dictates otherwise. Thus, for example, reference to a marker can include multiple markers unless the context clearly dictates otherwise.

As used herein, the terms “substantially,” “substantial,” and “about” are used to describe and account for small variations. When used in conjunction with an event or circumstance, the terms can refer to instances in which the event or circumstance occurs precisely as well as instances in which the event or circumstance occurs to a close approximation. For example, the terms can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations, which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scopes of this invention.

1. A method of treating a Pseudomonas aeruginosa infection in a subject, the method comprising administering to the subject an antiserum against Pf-family bacteriophage, wherein the antiserum contains anti-CoaB antibodies.

2. The method of claim 1, wherein the antiserum against Pf-family bacteriophage is made by administering to a subject a vaccine against Pf-family bacteriophage, the vaccine comprising (a) an immunogenic fragment of CoaB protein of Pf-family bacteriophage consisting of the amino acid sequence of GVIDTSAVESAITDGQGDM (SEQ ID NO: 1) conjugated to an immunogenic protein, and (b) a pharmaceutically acceptable carrier.

3. The method of claim 1, wherein the method reduces or prevents Pseudomonas aeruginosa biofilm formation in the subject.

4. The method of claim 1, wherein the subject is suffering from cystic fibrosis, burns, chronic would, chronic rhinosinusitis, ventilator-associated pneumonia, catheter-associated urinary tract infections, septic shock, and/or gastrointestinal infections.

5. The method of claim 1, further comprising administering one or more secondary antibodies to the subject.

6. The method of claim 5, wherein the secondary antibodies comprise IgG or IgM antibodies.

7. An antigen-binding polypeptide that binds specifically to a CoaB protein of Pf-family bacteriophage or fragment thereof.

8. The antigen-binding polypeptide of claim 7, wherein the antigen-binding polypeptide specifically binds to an antigenic fragment of the CoaB protein consisting of the amino acid sequence of GVIDTSAVESAITDGQGDM (SEQ ID NO: 1).

9. The antigen-binding polypeptide of claim 7, wherein the antigen-binding polypeptide comprises:
(a) a heavy chain variable region comprising:
(1) the CDR-H1 comprising the amino acid sequence of GFTFSSYV (SEQ ID NO: 6);
(2) the CDR-H2 comprising the amino acid sequence of ISSGGST (SEQ ID NO: 7); and
(3) the CDR-H3 comprising the amino acid sequence of LRGQDYGAAY (SEQ ID NO: 8),
a heavy chain variable region comprising:
(1) the CDR-H1 comprising the amino acid sequence of GYSFTSYW (SEQ ID NO: 16);
(2) the CDR-H2 comprising the amino acid sequence of TYPGNSDT (SEQ ID NO: 17); and
(3) the CDR-H3 comprising the amino acid sequence of TRSQFYSGSSEDAMDY (SEQ ID NO: 18), or
a heavy chain variable region comprising:
(1) the CDR-H1 comprising the amino acid sequence of GYTFTNYG (SEQ ID NO: 26);
(2) the CDR-H2 comprising the amino acid sequence of INTNTGEP (SEQ ID NO: 27); and
(3) the CDR-H3 comprising the amino acid sequence of ARKDYRYWFAY (SEQ ID NO: 28); and
(b) a light chain variable region comprising:
(1) the CDR-L1 comprising the amino acid sequence of QSLLDSDGKTY (SEQ ID NO: 9);
(2) the CDR-L2 comprising the amino acid sequence of LVS (SEQ ID NO: 10); and
(3) the CDR-L3 comprising the amino acid sequence of WQGTHEPOT (SEQ ID NO: 11),
a light chain variable region comprising:
(1) the CDR-L1 comprising the amino acid sequence of QSIVHSNGNTY (SEQ ID NO: 19);
(2) the CDR-L2 comprising the amino acid sequence of KVS (SEQ ID NO: 20); and
(3) the CDR-L3 comprising the amino acid sequence of FOGSHVPWT (SEQ ID NO: 21), or
a light chain variable region comprising:
(1) the CDR-L1 comprising the amino acid sequence of QSIVHSNGNTY (SEQ ID NO: 29);
(2) the CDR-L2 comprising the amino acid sequence of KVS (SEQ ID NO: 30); and
(3) the CDR-L3 comprising the amino acid sequence of FOGSHVPFT (SEQ ID NO: 31).

10. The antigen-binding polypeptide of claim 7, wherein the antigen-binding polypeptide is a monoclonal antibody, a chimeric antibody, a humanized antibody, a Fab fragment, a Fab′ fragment, a F(ab′) 2 fragment, or an scFv molecule.

11. A pharmaceutical composition comprising (a) the antigen-binding polypeptide of claim 7 or a fusion molecule comprising the antigen-binding polypeptide of claim 7, and (b) a pharmaceutically acceptable carrier.

12. A polynucleotide encoding the antigen-binding polypeptide of claim 7.

13. An expression cassette comprising a promoter operably linked to the polynucleotide of claim 12.

14. A vector comprising the expression cassette of claim 13.

15. A transformed cell comprising the expression cassette of claim 13.

16. A vaccine against Pf-family bacteriophage, comprising (a) an immunogenic fragment of CoaB protein of Pf-family bacteriophage consisting of the amino acid sequence of GVIDTSAVESAITDGQGDM (SEQ ID NO: 1), conjugated to an immunogenic protein and (b) a pharmaceutically acceptable carrier.

17. An antigen-binding polypeptide that binds specifically to a CoaB protein of Pf-family bacteriophage or fragment thereof.

18. The antigen-binding polypeptide of claim 17, wherein the antigen-binding polypeptide specifically binds to an antigenic fragment of the CoaB protein comprising the amino acid sequence of GVIDTSAVESAITDGQGDM (SEQ ID NO: 1).

19. The antigen-binding polypeptide of claim 17, wherein the antigen-binding polypeptide comprises:
(a) a heavy chain variable region comprising (1) the CDR-H1 comprising, consisting essentially of or consisting of the amino acid sequence of GFTFSSYV (SEQ ID NO: 6); (2) the CDR-H2 comprising, consisting essentially of or consisting of the amino acid sequence of ISSGGST (SEQ ID NO: 7); and (3) the CDR-H3 comprising, consisting essentially of or consisting of the amino acid sequence of LRGQDYGAAY (SEQ ID NO: 8),
a heavy chain variable region comprising (1) the CDR-H1 comprising, consisting essentially of or consisting of the amino acid sequence of GYSFTSYW (SEQ ID NO: 16; (2) the CDR-H2 comprising, consisting essentially of or consisting of the amino acid sequence of IYPGNSDT (SEQ ID NO: 17); and (3) the CDR-H3 comprising, consisting essentially of or consisting of the amino acid sequence of TRSQFYSGSSEDAMDY (SEQ ID NO: 18), or
a heavy chain variable region comprising (1) the CDR-H1 comprising, consisting essentially of or consisting of the amino acid sequence of GYTFTNYG (SEQ ID NO: 26; (2) the CDR-H2 comprising, consisting essentially of or consisting of the amino acid sequence of INTNTGEP (SEQ ID NO: 27); and (3) the CDR-H3 comprising, consisting essentially of or consisting of the amino acid sequence of ARKDYRYWFAY (SEQ ID NO: 28); and
(b) a light chain variable region comprising (1) the CDR-L1 comprising, consisting essentially of or consisting of the amino acid sequence of QSLLDSDGKTY (SEQ ID NO: 9); (2) the CDR-L2 comprising, consisting essentially of or consisting of the amino acid sequence of LVS (SEQ ID NO: 10); and (3) the CDR-L3 comprising, consisting essentially of or consisting of the amino acid sequence of WQGTHFPQT (SEQ ID NO: 11),
a light chain variable region comprising (1) the CDR-L1 comprising, consisting essentially of or consisting of the amino acid sequence of QSIVHSNGNTY (SEQ ID NO: 19); (2) the CDR-L2 comprising, consisting essentially of or consisting of the amino acid sequence of KVS (SEQ ID NO: 20); and (3) the CDR-L3 comprising, consisting essentially of or consisting of the amino acid sequence of FOGSHVPWT (SEQ ID NO: 21), or
a light chain variable region comprising (1) the CDR-L1 comprising, consisting essentially of or consisting of the amino acid sequence of QSIVHSNGNTY (SEQ ID NO: 29); (2) the CDR-L2 comprising, consisting essentially of or consisting of the amino acid sequence of KVS (SEQ ID NO: 30); and (3) the CDR-L3 comprising, consisting essentially of or consisting of the amino acid sequence of FOGSHVPFT (SEQ ID NO: 31).

20. The antigen-binding polypeptide of claim 17, wherein the antigen-binding polypeptide is a monoclonal antibody, a chimeric antibody, a humanized antibody, a Fab fragment, a Fab′ fragment, a F(ab′) 2 fragment, or an scFv molecule.


SEQUENCE LISTING (31 sequences)

SEQ ID NO: 1 - protein, 19 aa, Pseudomonas aeruginosa
GVIDTSAVESAITDGQGDM

SEQ ID NO: 2 - protein, 116 aa, Mus musculus
EVKLVESGGDLVKPGGSLKLSCAASGFTFSSYVMSWVRQTPEKRLEWVASISSGGSTYYPDSVKGRFTISRDNARNILYLQMSSLRSEDTAMYYCLRGQDYGAAYWGQGTLVTVSA

SEQ ID NO: 3 - protein, 348 aa, Mus musculus
GAAGTGAAGCTGGTGGAGTCTGGGGGAGACTTAGTGAAGCCTGGAGGGTCCCTGAAACTCTCCTGTGCAGCCTCTGGATTCACTTTCAGTAGCTATGTCATGTCTTGGGTTCGCCAGACTCCAGAAAAGAGGCTGGAGTGGGTCGCATCCATTAGTAGTGGTGGTAGCACCTACTATCCAGACAGTGTGAAGGGCCGATTCACCATCTCCAGAGATAATGCCAGGAACATCCTGTACCTGCAAATGAGTAGTCTGAGGTCTGAGGACACGGCCATGTATTACTGTTTAAGAGGCCAGGACTACGGCGCCGCTTACTGGGGCCAAGGGACTCTGGTCACTGTCTCTGCA

SEQ ID NO: 4 - protein, 112 aa, Mus musculus
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 5 - DNA, 336 bp, Mus musculus
  source 1..336: mol_type = other DNA
gatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcaaa

SEQ ID NO: 6 - protein, 8 aa, Mus musculus
GFTFSSYV

SEQ ID NO: 7 - protein, 7 aa, Mus musculus
ISSGGST

SEQ ID NO: 8 - protein, 10 aa, Mus musculus
LRGQDYGAAY

SEQ ID NO: 9 - protein, 11 aa, Mus musculus
QSLLDSDGKTY

SEQ ID NO: 10 - skipped

SEQ ID NO: 11 - protein, 9 aa, Mus musculus
WQGTHFPQT

SEQ ID NO: 12 - protein, 123 aa, Mus musculus
EVQLQQSGTVLARPGASVKMSCKASGYSFTSYWMHWVKQRPGQGLEWIGAIYPGNSDTSYNQKFKGKAKLTAVTSASTAYMELSCLTNEDSAVFYCTRSQFYSGSSEDAMDYWGQGTSVTVSS

SEQ ID NO: 13 - DNA, 369 bp, Mus musculus
  source 1..369: mol_type = other DNA
gaggttcagctccagcagtctgggactgtgctggcaaggcctggggcttccgtgaagatgtcctgcaaggcttctggctacagctttaccagctactggatgcactgggtaaaacagaggcctggacagggtctagaatggattggtgctatttatcctggaaatagtgatactagttacaaccagaagttcaagggcaaggccaaactgactgcagtcacatccgccagcactgcctacatggagctcagctgcctgacaaatgaggactctgcggtcttttactgtacaagatcccaattttactccggtagtagcgaggatgctatggactactggggtcaaggaacctcagtcaccgtctcctca

SEQ ID NO: 14 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYFCFQGSHVPWTFGGGTKLEIK

SEQ ID NO: 15 - DNA, 336 bp, Mus musculus
  source 1..336: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttatttctgctttcaaggttcacatgttccgtggacgttcggtggaggcaccaagctggaaatcaaa

SEQ ID NO: 16 - protein, 8 aa, Mus musculus
GYSFTSYW

SEQ ID NO: 17 - protein, 8 aa, Mus musculus
IYPGNSDT

SEQ ID NO: 18 - protein, 16 aa, Mus musculus
TRSQFYSGSSEDAMDY

SEQ ID NO: 19 - protein, 11 aa, Mus musculus
QSIVHSNGNTY

SEQ ID NO: 20 - skipped

SEQ ID NO: 21 - protein, 9 aa, Mus musculus
FQGSHVPWT

SEQ ID NO: 22 - protein, 118 aa, Mus musculus
QIQLVQSGPELKKPGETVKISCKASGYTFTNYGMNWLKQAPGKGLKWMGWINTNTGEPTYAEEFKGRFAFSLETSASTAYLQINNLKNEDTATYFCARKDYRYWFAYWGQGTLVTVSA

SEQ ID NO: 23 - DNA, 354 bp, Mus musculus
  source 1..354: mol_type = other DNA
cagatccagttggtgcagtctggacctgagctgaagaagcctggagagacagtcaagatctcctgcaaggcttctgggtataccttcacaaactatggaatgaactggctgaagcaggctccaggaaagggtttaaagtggatgggctggataaacaccaacactggagagccaacatatgctgaagagttcaagggacggtttgccttctctttggaaacctctgccagcactgcctatttgcagatcaacaacctcaaaaatgaggacacggctacatatttctgtgcaagaaaggactataggtactggtttgcttactggggccaagggactctggtcactgtctctgca

SEQ ID NO: 24 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 25 - DNA, 336 bp, Mus musculus
  source 1..336: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcggggacaaagttggaaataaaa

SEQ ID NO: 26 - protein, 8 aa, Mus musculus
GYTFTNYG

SEQ ID NO: 27 - protein, 8 aa, Mus musculus
INTNTGEP

SEQ ID NO: 28 - protein, 11 aa, Mus musculus
ARKDYRYWFAY

SEQ ID NO: 29 - protein, 11 aa, Mus musculus
QSIVHSNGNTY

SEQ ID NO: 30 - skipped

SEQ ID NO: 31 - protein, 9 aa, Mus musculus
FQGSHVPFT